(19) United States
(12) Patent Application Publication
(10) Pub. No.: US 2006/0110349 A1
(43) Pub. Date: May 25, 2006

(54) Hair care composition

(76) Inventors: Peter Gallagher, Chicago, IL (US); Ezat Khosdel, Wirrai (GB); Sheila Anne Ward, London (GB)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/521,982

(22) PCT Filed: Jul. 3, 2003
(86) PCT No.: PCT/EP03/07122

§ 371 Date: Dec. 8, 2005

(30) Foreign Application Priority Data
Jul. 22, 2002 (EP) 02255129.5

(51) Int. Cl. A61K 8/81; A61K 8/365

(52) U.S. Cl. 424/70.11; 424/70.22

(57) ABSTRACT

A hair treatment composition comprising (i) 2-hydroxyalkanoic acid and; (ii) 0.1 to 10 wt % of a styling aid.

### FIELD OF THE INVENTION

This invention relates to hair care compositions, in particular to hair care compositions that style the hair.

### BACKGROUND AND PRIOR ART

The desire to have the hair retain a particular shape or style is widely held. The most common approach for accomplishing styling of hair is the application of a composition to dampened hair, after shampooing and/or conditioning, or to dry, styled hair. These compositions provide temporary styling benefits and can readily be removed by water or shampooing. To date, the materials employed in hair care compositions to provide styling benefits have generally been natural or synthetic resins such as polymers of acrylate and polymers of acrylate with grafted silicone copolymers. Styling compositions are usually applied in the form of, sprays, mousses, gels and lotions.

Shampoo compositions comprising 2-hydroxyalkanoic acids are disclosed in EP 0 403 304 and EP 0 424 158. The 2-hydroxyalkanoic acid is said to enhance the elasticity and the colour penetration of hair respectively.

Frequently styled hair drops or loses its shape when subjected to high humidity, thus there is a need for styling products that address this problem.

### DESCRIPTION OF THE PRESENT INVENTION

According to the present invention, there is provided a hair treatment composition comprising

i) 2-hydroxyalkanoic acid and;

ii) 0.1 to 10 wt % of a styling aid.

The invention also relates to a method of styling hair by applying to the hair a composition comprising a 2-hydroxyalkanoic acid.

In a further aspect, the invention provides the use of a 2-hydroxyalkanoic acid to style and to impart humidity resistance to hair.

### DETAILED DESCRIPTION OF THE INVENTION

### 2-hydroxy alkanoic acids

The compositions of the invention comprise as an essential element a 2-hydroxy alkanoic acid. It is preferred if the 2-hydroxy alkanoic acids have an unbranched alkyl chain. It is also advantageous if the alkyl chain has 12 or less carbon atoms.

Particularly preferred 2-hydroxyalkanoic acids are selected from the group consisting of 2-hydroxyhexanoic acid 2-hydroxyoctanoic acid, 2 hydroxy nonanoic, 2-hydroxydecanoic acid or mixtures thereof. Hydroxyoctanoic acid is especially preferred.

Styling Compound

In many aspects of this invention it is highly desirable if the composition comprises a styling aid.

Particularly useful as styling aids with this invention are hair styling polymers. Hair styling polymers are well known articles of commerce and many such polymers are available commercially which contain moieties which render the polymers cationic, anionic, amphoteric or nonionic in nature. The polymers may be synthetic or naturally derived.

Styling aids such as vinylic polymer are preferred, in particular block copolymers.

The amount of the hair styling polymer may range from 0.1 to 10%, preferably 0.5 to 8%, more preferably 0.75 to 6% by weight based on total weight of the composition.

Examples of anionic hair styling polymers are:

copolymers of vinyl acetate and crotonic acid; terpolymers of vinyl acetate, crotonic acid and a vinyl ester of an alpha-branched saturated aliphatic monocarboxylic acid such as vinyl neodecanoate; copolymers of methyl vinyl ether and maleic anhydride (molar ratio about 1:1) wherein such copolymers are 50% esterified with a saturated alcohol containing from 1 to 4 carbon atoms such as ethanol or butanol;

acrylic copolymers containing acrylic acid or methacrylic acid as the anionic radical-containing moiety with other monomers such as: esters of acrylic or methacrylic acid with one or more saturated alcohols having from 1 to 22 carbon atoms (such as methyl methacrylate, ethyl acrylate, ethyl methacrylate, n-butyl acrylate, t-butyl acrylate, t-butyl methacrylate, n-butyl methacrylate, n-hexyl acrylate, n-octyl acrylate, lauryl methacrylate and behenyl acrylate); glycols having from 1 to 6 carbon atoms (such as hydroxypropyl methacrylate and hydroxyethyl acrylate); styrene; vinyl caprolactam; vinyl acetate; acrylamide; alkyl acrylamides and methacrylamides having 1 to 8 carbon atoms in the alkyl group (such as methacrylamide, t-butyl acrylamide and n-octyl acrylamide); and other compatible unsaturated monomers.

The polymer may also contain grafted silicone, such as polydimethylsiloxane.

Specific examples of suitable anionic hair styling polymers are:

RESYN® 28-2930 available from National Starch (vinyl acetate/crotonic acid/vinyl neodecanoate copolymer);

ULTRAHOLD® 8 available from BASF (CTFA designation Acrylates/acrylamide copolymer);

the GANTREZ®ES series available from ISP corporation (esterified copolymers of methyl vinyl ether and maleic anhydride).

Other suitable anionic hair styling polymers include carboxylated polyurethanes. Carboxylated polyurethane resins are linear, hydroxyl-terminated copolymers having pendant carboxyl groups. They may be ethoxylated and/or propoxylated at least at one terminal end. The carboxyl group can be a carboxylic acid group or an ester group, wherein the alkyl moiety of the ester group contains one to three carbon atoms. The carboxylated polyurethane resin can also be a copolymer of polyvinylpyrrolidone and a polyurethane, having a CTFA designation PVP/polycarbamyl polyglycol ester. Suitable carboxylated polyurethane resins are disclosed in EP-A-0619111 and U.S. Pat. No. 5,000,955. Other suitable hydrophilic polyurethanes are disclosed in U.S. Pat. Nos. 3,822,238; 4,156,066; 4,156,067; 4,255,550; and 4,743,673.

Amphoteric hair styling polymers which can contain cationic groups derived from monomers such as t-butyl aminoethyl methacrylate as well as carboxyl groups derived from monomers such as acrylic acid or methacrylic acid can also be used in the present invention. One specific example of an amphoteric hair styling polymer is Amphomer® (Octylacrylamide/acrylates/butylaminoethyl methacrylate copolymer) sold by the National Starch and Chemical Corporation.

Examples of nonionic hair styling polymers are homopolymers of N-vinylpyrrolidone and copolymers of N-vinylpyrrolidone with compatible nonionic monomers such as vinyl acetate. Nonionic polymers containing N-vinylpyrrolidone in various weight average molecular weights are available commercially from ISP Corporation—specific examples of such materials are homopolymers of N-vinylpyrrolidone having an average molecular weight of about 630,000 sold under the name PVP K-90 and are homopolymers of N-vinylpyrrolidone having an average molecular weight of about 1,000,000 sold under the name of PVP K-120.

Other suitable nonionic hair styling polymers are cross-linked silicone resins or gums. Specific examples include rigid silicone polymers such as those described in EP-A-0240350 and cross-linked silicone gums such as those described in WO 96/31188.

Examples of cationic hair styling polymers are copolymers of amino-functional acrylate monomers such as lower alkyl aminoalkyl acrylate, or methacrylate monomers such as dimethylaminoethyl methacrylate, with compatible monomers such as N-vinylpyrrolidone, vinyl caprolactam, alkyl methacrylates (such as methyl methacrylate and ethyl methacrylate) and alkyl acrylates (such as ethyl acrylate and n-butyl acrylate).

Specific examples of suitable cationic hair styling polymers are:

copolymers of N-vinylpyrrolidone and dimethylaminoethyl methacrylate, available from ISP Corporation as Copolymer 845, Copolymer 937 and Copolymer 958;

copolymers of N-vinylpyrrolidone and dimethylaminopropylacrylamide or methacrylamide, available from ISP Corporation as Styleze® CC10;

copolymers of N-vinylpyrrolidine and dimethylaminoethyl methacrylate;

copolymers of vinylcaprolactam, N-vinylpyrrolidone and dimethylaminoethylmethacrylate;

Polyquaternium-4 (a copolymer of diallyldimonium chloride and hydroxyethylcellulose);

Polyquaternium-11 (formed by the reaction of diethyl sulphate and a copolymer of vinyl pyrrolidone and dimethyl aminoethylmethacrylate), available from ISP as Gafquat® 734, 755 and 755N, and from BASF as Luviquat® PQ11;

Polyquaternium-16 (formed from methylvinylimidazolium chloride and vinylpyrrolidone), available from BASF as Luviquat® FC 370, FC 550, FC 905 and HM-552;

Polyquaternium-46 (prepared by the reaction of vinylcaprolactam and vinylpyrrolidone with methylvinylimidazolium methosulphate), available from BASF as Luviquat®Hold.

Examples of suitable naturally-derived hair styling polymers include shellac, alginates, gelatins, pectins, cellulose derivatives and chitosan or salts and derivatives thereof. Commercially available examples include Kytamer® (ex Amerchol) and Amaze® (ex National Starch).

Also suitable for use as optional components in the compositions of the invention are the ionic copolymers described in WO 93/03703, the polysiloxane-grafted polymers disclosed in WO 93/23446, the silicone-containing polycarboxylic acid copolymers described in WO 95/00106 or WO 95/32703, the thermoplastic elastomeric copolymers described in WO 95/01383, WO 95/06078, WO 95/06079 and WO 95/01384, the silicone grafted adhesive polymers disclosed in WO 95/04518 or WO 95/05800, the silicone macro-grafted copolymers taught in WO 96/21417, the silicone macromers of WO 96/32918, the adhesive polymers of WO 98/48770 or WO 98/48771 or WO 98/48772 or WO 98/48776, the graft polymers of WO 98/51261 and the grafted copolymers described in WO 98/51755.

With certain of the above-described polymers it may be necessary to neutralise some acidic groups to promote solubility/dispersibility. Examples of suitable neutralising agents include 2-amino-2-methyl-1,3-propanediol (AMPD); 2-amino-2-ethyl-1,3-propanediol (AEPD); 2-amino-2-methyl-1-propanol (AMP); 2-amino-1-butanol (AB); monoethanolamine (MEA); diethanolamine (DEA); triethanolamine (TEA); monoisopropanolamine (MIPA); diisopropanol-amine (DIPA); triisopropanolamine (TIPA); and dimethyl stearamine (DMS). A long chain amine neutralising agent such as stearamidopropyl dimethylamine or lauramidopropyl dimethylamine may be employed, as is described in U.S. Pat. No. 4,874,604. Also suitable are inorganic neutralisers, examples of which include sodium hydroxide, potassium hydroxide and borax. Mixtures of any of the above neutralising agents may be used. Amounts of the neutralising agents will range from about 0.001 to about 10% by weight of the total composition.

Product Type

Compositions of the present invention are formulated into hair care compositions, especially products with hair styling claims. The compositions are for use in styling human hair and, more preferably, they are packaged and labelled as such.

It is preferred if the products are left on hair after application and not immediately washed off.

Further Components

Such styling products frequently include a carrier and further additional components. The carriers and additional components required to formulate such products vary with product type and can be routinely chosen by one skilled in the art. The following is a description of some of these carriers and additional components.

Hair care compositions of the present invention can comprise a carrier, or a mixture of such carriers, which are suitable for application to the hair. The carriers are present at from about 0.5% to about 99.5%, preferably from about 5.0% to about 99.5%, more preferably from about 10.0% to about 98.0%, of the composition. As used herein, the phrase “suitable for application to hair” means that the carrier does not damage or negatively affect the aesthetics of hair or cause irritation to the underlying skin.

Compositions according to the invention comprise a buffer or pH adjuster. Preferred buffers or pH adjusters include weak acids and bases such glycine/sodium hydroxide, citric acid, lactic acid, succinic acid, acetic salt and salts thereof. Frequently a mixture of buffering system is used such as sodium citrate and citric acid.

Carriers suitable for use with hair care compositions of the present invention include, for example, those used in the formulation of hair sprays, mousses, tonics, gels, shampoos, conditioners, and rinses. The choice of appropriate carrier will depend on the particular product to be formulated. The carriers used herein can include a wide range of components conventionally used in hair care compositions. The carriers can contain a solvent to dissolve or disperse the styling compound being used, with water, the C₁-C₆ alcohols, lower alkyl acetate and mixtures thereof being preferred. The carriers can also contain a wide variety of additional materials such as acetone, hydrocarbons (such as isobutane, hexane, decene), halogenated hydrocarbons (such as Freons) and volatile silicones such as cyclomethicone.

When the hair care composition is a hair spray, tonic, gel, or mousse the preferred solvents include water, ethanol, volatile silicone derivatives, and mixtures thereof. The solvents used in such mixtures may be miscible or immiscible with each other. Mousses and aerosol hair sprays can also utilise any of the conventional propellants to deliver the material as a foam (in the case of a mousse) or as a fine, uniform spray (in the case of an aerosol hair spray). Examples of suitable propellants include materials such as trichlorofluoromethane, dichlorodifluoromethane, difluoroethane, dimethylether, propane, n-butane or isobutane. A tonic or hair spray product having a low viscosity may also utilise an emulsifying agent. Examples of suitable emulsifying agents include nonionic, cationic, anionic surfactants, or mixtures thereof. If such an emulsifying agent is used, it is preferably present at a level of from about 0.01% to about 7.5% by weight based on total weight of the composition. The level of propellant can be adjusted as desired but is generally from about 3% to about 30% by weight based on total weight for mousse compositions and from about 15% to about 50% by weight based on total weight for aerosol hair spray compositions.

Hair styling creams or gels also typically contain a structurant or thickener, typically in an amount of from 0.01% to 10% by weight.

Suitable spray containers are well known in the art and include conventional, non-aerosol pump sprays i.e., “atomisers”, aerosol containers or cans having propellant, as described above, and also pump aerosol containers utilising compressed air as the propellant.

Where the hair care compositions are conditioners and rinses, the carrier can include a wide variety of conditioning materials such as cationic conditioners suitable for hair, quaternary silicone polymers, silicone based conditioners and their emulsions, and amino functional silicones and their emulsions.

Where the hair care compositions are shampoos, the carrier can include, for example, anionic surfactants, nononic surfactants, amphoteric surfactants, suspending agents, and thickeners

Further general ingredients suitable for all product forms include, sun-screening agents, anti-dandruff actives, carboxylic acid polymer thickeners for hair shampoo and conditioner compositions and emulsifiers for emulsifying the various carrier components of the compositions of the invention.

In hair treatment compositions containing a conditioning agent, it is preferred that a cationic polymer also be present.

The compositions of the present invention may also contain adjuncts suitable for hair care. Generally such ingredients are included individually at a level of up to 2, preferably up to 1 wt % of the total composition. Suitable hair care adjuncts, include amino acids, sugars and ceramides.

The method of the invention comprises applying 2-hydroxyalkanoic acid or mixtures thereof to the hair during or immediately before styling. The compound is preferably in the form of a composition of the invention when it is applied to the hair, although other product forms may also be used, such as for example a simple solution of the compound.

The following non-limiting Examples further illustrate the preferred embodiments of the invention. All percentages referred to in the examples and throughout this specification are by weight based on total weight unless otherwise indicated.

### EXAMPLES

### Examples 1 to 7

### Compositions of the Invention

The following are examples of compositions of the invention.

The materials in the examples include the following:

| Material | Chemical Name | Supplier |
|---|---|---|
| Silicone emulsion X2 1787 ™ | | Dow Corning |
| VOLPO CS 50 ™ | | Croda Chemicals |
| Silcone DC 200/DC 24 S ™ | | Dow Corning |
| Silwet L7602/L-720 ™ | | Union Carbide |
| CAP 40 ™ | | Calor Gas |
| Carbopol 980 ™ | | BF Goodrich |
| Jaguar HP-105 ™ | | Rhodia |
| Silicone Fluid 245 ™ | | Dow Corning |
| Gafquat 734 (trademark) | Polyquaternium-11 | ISP |
| PVP K-120 | | ISP |
| Amphomer 284910 | | National starch |
| Ethanol is SD Alcohol 40-B | | |
| (92% active) | | |

### Example 1

A styling mousse is formulated as follows:

| Material | % in product (w/w) |
|---|---|
| Silicone Emulsion X2 1787 | 1.2 |
| Hydroxyoctanoic acid | 1.5 |
| Gafquat 734 | 2.0 |
| VOLPO CS 50 | 0.3 |
| Sepicide LD | 0.4 |
| Cremophor RH410 | 0.2 |
| Ethanol | 7.5 |
| CAP 40 | 8.0 |
| Perfume | 0.2 |
| Water | to 100% |

### Example 2

A hairspray is formulated as follows:

| Material | % in product (w/w) |
|---|---|
| Hydroxyoctanoic acid | 1.3 |
| Amphomer 284910 | 3.0 |
| Silicone DC200 | 0.09 |
| Silwet L7602 | 0.09 |
| CAP 40 | 35.0 |
| Ethanol | 60.0 |
| Perfume | 0.10 |
| Water | to 100% |

### Example 3

A pump spray is formulated as follows:

| Material | % w/w |
|---|---|
| Amphomer | 3.5 |
| Silwet L-720 | 0.3 |
| Silicone DC24S | 0.15 |
| Fragrance | 0.3 |
| Water | to 100% |
| Ethanol | 60.0 |
| Hydroxyoctanoic acid | 1.0 |

### Example 4

A styling gel is formulated as follows:

| Material | % w/w |
|---|---|
| Hydroxyoctanoic acid | 0.9 |
| PVP | 3.8 |
| Carbopol 980 | 0.4 |
| Water | to 100% |
| Sepicide LD | 0.4 |
| Sodium hydroxide (8% 2M) | 0.1 |
| Ethanol | 10.0 |
| Cremaphor RH410 | 0.4 |
| Jaguar HP-105 | 0.2 |
| Perfume | 0.15 |

### Example 5

A 55% voc propelled aerosol composition is formulated as follows:

| Material | % w/w |
|---|---|
| Hydroxyoctanoic acid | 0.6 |
| Amphomer | 3.0 |
| Silicone Fluid 245 | 0.20 |
| Fragrance | 0.32 |
| Ethanol | 19.53 |
| Dimethyl ether | 35.00 |
| Sodium benzoate | 0.26 |
| Cyclohexylamine | 0.21 |
| Water | to 100% |

### Example 6

A 55% voc pump hairspray composition is formulated as follows:

| Material | % w/w |
|---|---|
| Hydroxyoctanoic acid | 0.6 |
| Amphomer | 3.0 |
| Cyclopentasiloxane (99% active) | 0.15 |
| Benzophenone 4 | 0.0001 |
| Fragrance | 0.25 |
| Ethanol | 58.00 |
| Water | to 100% |

### Example 7

Two solutions were formulated containing 0.5 wt % and 1 wt % by weight of 2 hydroxy octanoic acid. Hair curls were made up using perming rods and 30 cm length hair.

The curls were placed in the solution for 1 hour, rinsed thoroughly with distilled water and dried for 30 minutes. The hair was removed from the curling rod and the length of the curl measured.

The hair was hung in high humidity environmental chamber (90% RH, 30° C.) with fan agitation for 1 minute and measured again.

| | Styling | | | | Curl |
|---|---|---|---|---|---|
| Experiment | compound | Wt % | solvent | | dropout % |
| Negative | Water | 100% | Water | | 64 |
| Control | | | | | |
| Positive | Commercial | N/A | N/A | At least 2 | 45.4 |
| Control | Hairspray | | | actuations | |
| | | | | of spray | |
| | | | | applied to | |
| | | | | each side | |
| | | | | of curl | |
| Test | 2 hydroxy | 0.5% | Water | / | 33.6 |
| Compound | octanoic | | | | |
| | acid | | | | |
| Test | 2 hydroxy | 1% | Water | / | 17.6 |
| Compound | octanoic | | | | |
| | acid | | | | |

*Spray (John Freda Frizzease UK, 2000) applied after curl was treated and dried as negative control.

1. A hair treatment composition comprising
i) 2-hydroxyalkanoic acid and;
ii) 0.1 to 10 wt % of a styling aid.

2. A hair treatment composition according to claim 1 in which the 2-hydroxyalkanoinic acid is selected from the group consisting of 2-hydroxyhexanoic acid, 2 hydroxy heptanoic acid, 2-hydroxyoctanoic acid, 2-hydroxynonanoic acid, 2-hydroxydecanoic acid or mixtures thereof.

3. A hair treatment composition according to claim 1 in which the 2-hydroxyalkanoic acid is 2-hydroxyoctanoic acid.

4. A hair treatment composition according to claim 1 in which the styling aid is a vinylic polymer.

5. A hair treatment composition according to claim 1 in which the polymer is a block copolymer.

6. A hair treatment composition according to claim 1 that is a leave on product.

7. A method of styling hair by applying to the hair a composition comprising a 2-hydroxyalkanoic acid.

8. Use of a 2-hydroxyalkanoic acid to style hair.

9. Use of a 2-hydroxyalkanoic acid to impart a longer lasting hairstyle at high humidity.
